(19) United States
(12) Patent Application Publication
(10) Pub. No.: US 2018/0161175 A1
(43) Pub. Date: Jun. 14, 2018

(54) INTERVERTEBRAL IMPLANT INSERTER AND RELATED METHODS

(71) Applicant: DePuy Synthes Products, Inc., Raynham, MA (US)

(72) Inventors: William Frasier, New Bedford, MA (US); Sean Saidha, Franklin, MA (US); Thomas Martin, Riverside, RI (US); Paul Maguire, Hope Valley, RI (US)

(21) Appl. No.: 15/378,724

(22) Filed: Dec. 14, 2016

(51) Int. Cl. A61F 2/46; A61F 2/44

(52) U.S. Cl. CPC A61F 2/4611; A61F 2002/30579; A61F 2/447

(57) ABSTRACT

An insertion instrument is configured to attach and secure to an expandable implant. The insertion instrument includes a securement member that is configured to be secured to the implant both when the implant is in a collapsed configuration and when the implant is in an expanded configuration. The insertion instrument further includes a drive member that is configured to actuate the implant to the expanded configuration.

### TECHNICAL FIELD

The present invention relates to an expandable intervertebral implant, system, kit and method.

### BACKGROUND

The human spine is comprised of a series of vertebral bodies separated by intervertebral discs. The natural intervertebral disc contains a jelly-like nucleus pulposus surrounded by a fibrous annulus fibrosus. Under an axial load, the nucleus pulposus compresses and radially transfers that load to the annulus fibrosus. The laminated nature of the annulus fibrosus provides it with a high tensile strength and so allows it to expand radially in response to this transferred load.

In a healthy intervertebral disc, cells within the nucleus pulposus produce an extracellular matrix (ECM) containing a high percentage of proteoglycans. These proteoglycans contain sulfated functional groups that retain water, thereby providing the nucleus pulposus within its cushioning qualities. These nucleus pulposus cells may also secrete small amounts of cytokines such as interleukin-1.beta. and TNF-.alpha. as well as matrix metalloproteinases (“MMPs”). These cytokines and MMPs help regulate the metabolism of the nucleus pulposus cells.

In some instances of disc degeneration disease (DDD), gradual degeneration of the intervetebral disc is caused by mechanical instabilities in other portions of the spine. In these instances, increased loads and pressures on the nucleus pulposus cause the cells within the disc (or invading macrophases) to emit larger than normal amounts of the above-mentioned cytokines. In other instances of DDD, genetic factors or apoptosis can also cause the cells within the nucleus pulposus to emit toxic amounts of these cytokines and MMPs. In some instances, the pumping action of the disc may malfunction (due to, for example, a decrease in the proteoglycan concentration within the nucleus pulposus), thereby retarding the flow of nutrients into the disc as well as the flow of waste products out of the disc. This reduced capacity to eliminate waste may result in the accumulation of high levels of toxins that may cause nerve irritation and pain.

As DDD progresses, toxic levels of the cytokines and MMPs present in the nucleus pulposus begin to degrade the extracellular matrix, in particular, the MMPs (as mediated by the cytokines) begin cleaving the water-retaining portions of the proteoglycans, thereby reducing its water-retaining capabilities. This degradation leads to a less flexible nucleus pulposus, and so changes the loading pattern within the disc, thereby possibly causing delamination of the annulus fibrosus. These changes cause more mechanical instability, thereby causing the cells to emit even more cytokines, thereby upregulating MMPs. As this destructive cascade continues and DDD further progresses, the disc begins to bulge (“a herniated disc”), and then ultimately ruptures, which may cause the nucleus pulposus to contact the spinal cord and produce pain.

One proposed method of managing these problems is to remove the problematic disc and replace it with a device that restores disc height and allows for bone growth between the adjacent vertebrae. These devices are commonly called fusion devices, or “interbody fusion devices”. Current spinal fusion procedures include transforaminal lumbar interbody fusion (TLIF), posterior lumbar interbody fusion (PLIF), and extreme lateral interbody fusion (XLIF) procedures.

### SUMMARY

According to one embodiment of the present disclosure, an insertion instrument is configured to implant an expandable intervertebral implant in an intervertebral space. The insertion instrument can include a drive shaft elongate along a longitudinal direction, and a drive member disposed at a distal end of the drive shaft. The drive member can be configured to 1) couple to a complementary driven member of the implant, and 2) iterate the intervertebral implant from a collapsed configuration to an expanded configuration. The insertion instrument can further include a securement member that is spaced from the drive member along a lateral direction that is perpendicular to the longitudinal direction, the securement member having at least one guide rail that has a height along a transverse direction sufficient to 1) reside in a corresponding at least one guide channel of the implant when the implant is in the collapsed configuration, 2) ride along the implant in the at least one guide channel as the implant expands to the expanded configuration, and 3) remain in the corresponding at least one guide channel when the implant is in the expanded configuration. The transverse direction is perpendicular to each of the longitudinal direction and the lateral direction.

### BRIEF DESCRIPTION OF THE DRAWINGS

The foregoing summary, as well as the following detailed description of illustrative embodiments of the intervertebral implant of the present application, will be better understood when read in conjunction with the appended drawings. For the purposes of illustrating aspects of the present disclosure, there is shown in the drawings illustrative embodiments. It should be understood, however, that the disclosure is not limited to the precise arrangements and instrumentalities shown. In the drawings:

FIG. 1 is a perspective view of an expandable implant shown implanted in an intervertebral disc space, showing the implant in a collapsed position;

FIG. 2A is a perspective view of the expandable implant of FIG. 1;

FIG. 2B is a perspective view of the expandable implant of FIG. 2A, but shown in an expanded configuration;

FIG. 3 is an exploded perspective view of the expandable implant of FIG. 2A:

FIG. 4A is a side elevation view of an intervertebral implant system including the expandable implant of claim 1 and an insertion instrument configured to secure to and actuate the expandable implant;

FIG. 4B is a perspective view of the insertion instrument of FIG. 4A;

FIG. 4C is an exploded side elevation view of the insertion instrument of FIG. 4B;

FIG. 4D is an enlarged top plan view of a securement member of the insertion instrument of FIG. 4B;

FIG. 4E is an enlarged partial cut-away perspective view of a portion of the insertion instrument illustrated in FIG. 4C;

FIG. 5A is a sectional plan view of the insertion instrument aligned for securement with the expandable implant;

FIG. 5B is an enlarged sectional plan view of a portion of the insertion instrument and the expandable implant of FIG. 5A, taken at Region 5B;

FIG. 6A is a sectional plan view similar to FIG. 5A, but showing the insertion instrument attached to the expandable implant;

FIG. 6B is an enlarged sectional plan view of a portion of the insertion instrument and the expandable implant of FIG. 6A, taken at Region 6B;

FIG. 7A is a sectional plan view similar to FIG. 6A, but showing the insertion instrument secured to the expandable implant;

FIG. 7B is an enlarged sectional plan view of a portion of the insertion instrument and the expandable implant of FIG. 7A, taken at Region 7B;

FIG. 8A is a sectional plan view similar to FIG. 7A, but showing a drive member of the insertion instrument rotationally coupled to a driven member of the expandable implant;

FIG. 8B is an enlarged perspective view showing the insertion instrument secured to the expandable implant with the drive member of the insertion instrument coupled to the driven member of the expandable implant as illustrated in FIG. 7A, showing the implant in a collapsed configuration;

FIG. 9A is a sectional plan view similar to FIG. 8A, but after the insertion instrument has driven the implant to expand from the collapsed configuration to the expanded configuration;

FIG. 9B is an enlarged sectional plan view of a portion of the insertion instrument and the expandable implant of FIG. 9A, taken at Region 9B;

FIG. 9C is a perspective view of a portion of the instrument and expandable implant of FIG. 9A;

FIG. 10A is a sectional plan view similar to FIG. 9A, but showing the drive member of the insertion instrument decoupled from the driven member of the expandable implant;

FIG. 10B is an enlarged sectional plan view of a portion of the insertion instrument and the expandable implant of FIG. 10A, taken at Region 10B;

FIG. 11 is a sectional plan view similar to FIG. 10A, but after removal of the securement of the insertion instrument to the expandable implant, such that the insertion instrument is configured to be removed from the expandable implant.

### DETAILED DESCRIPTION OF ILLUSTRATIVE EMBODIMENTS

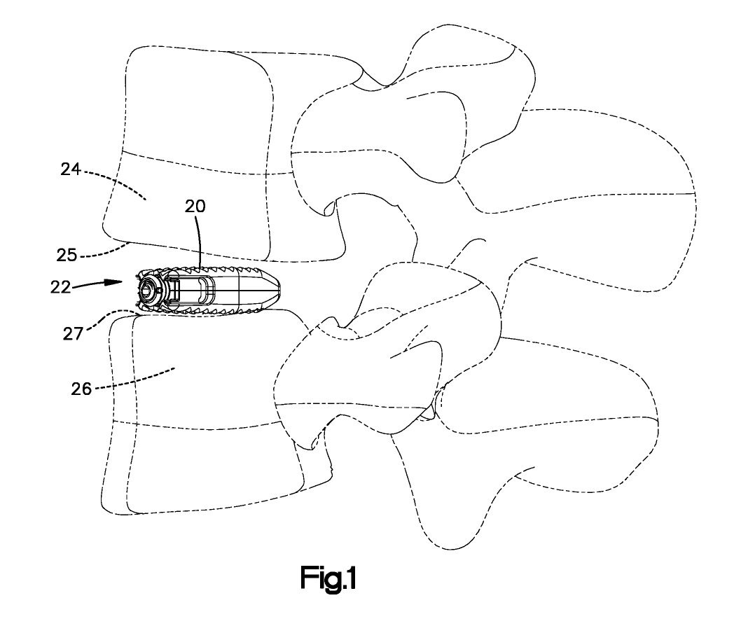

Referring initially to FIGS. 1-3, an expandable intervertebral implant 20 is configured for implantation in an intervertebral space 22 that is defined between a first or superior vertebral body 24 and a second or inferior vertebral body 26. The vertebral bodies 24 and 26 can be anatomically adjacent each other, or can be remaining vertebral bodies after a corpectomy procedure has removed a vertebral body from a location between the vertebral bodies 24 and 26. The intervertebral space 22 in FIG. 1 is illustrated after a discectomy, whereby the disc material has been removed or at least partially removed from the intervertebral space 22 to prepare the intervertebral space 22 to receive the intervertebral implant 20.

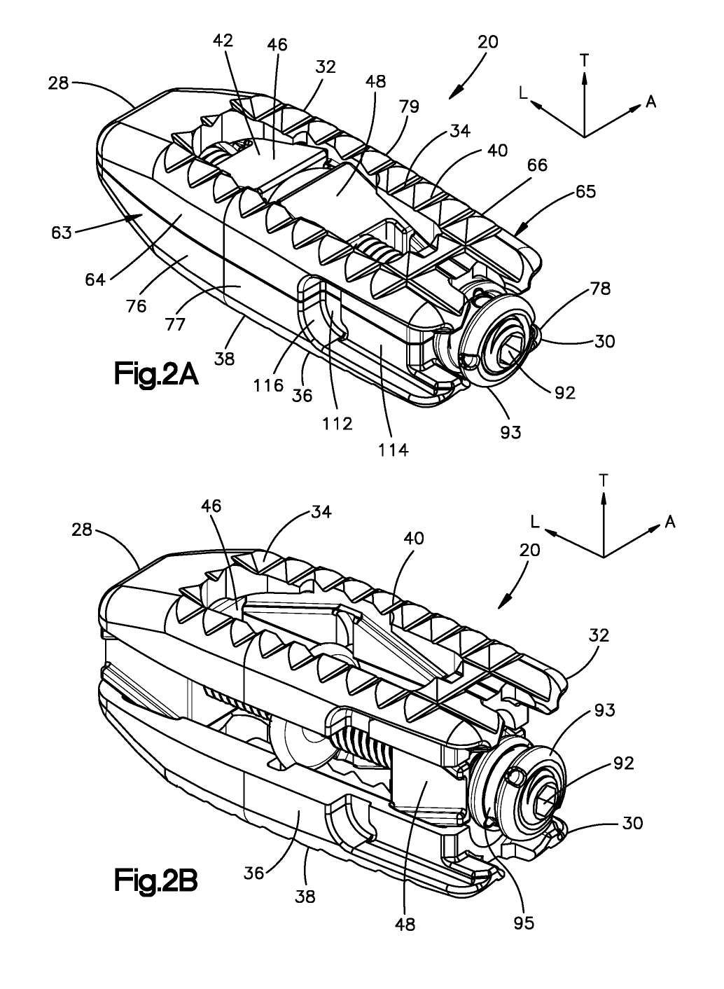

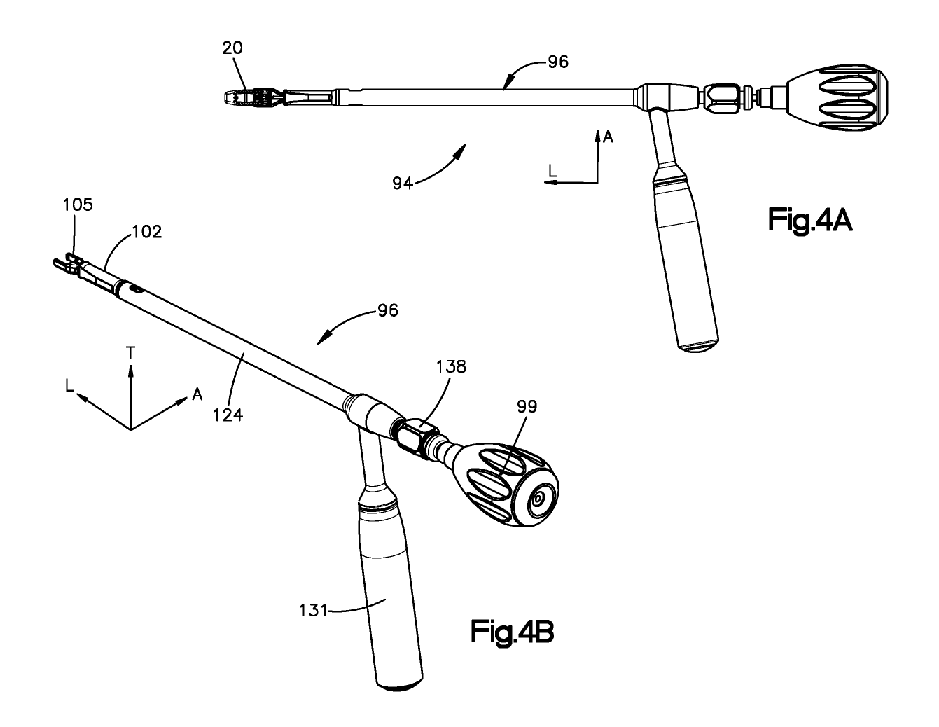

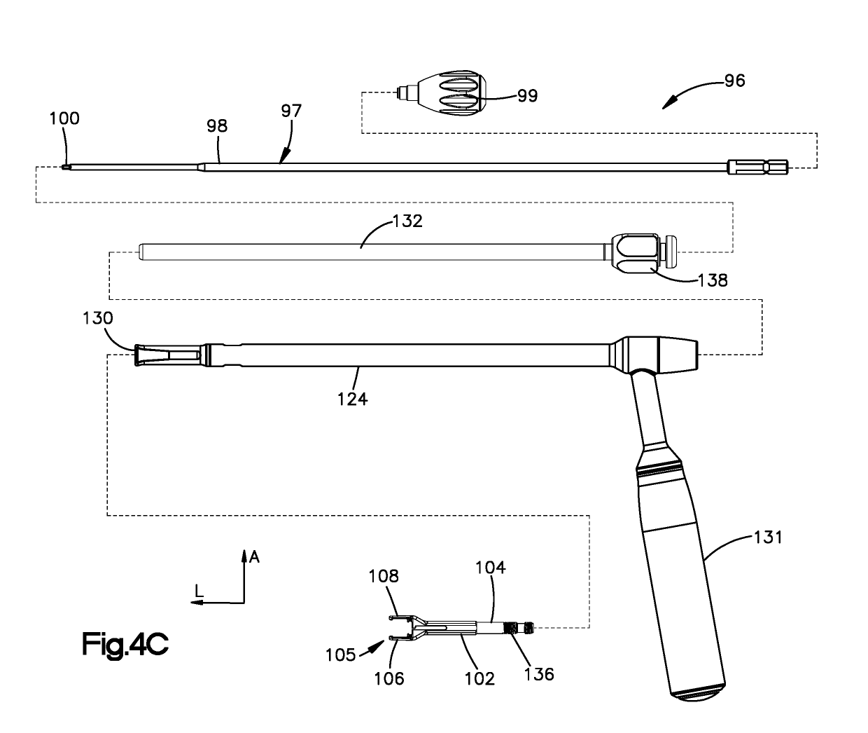

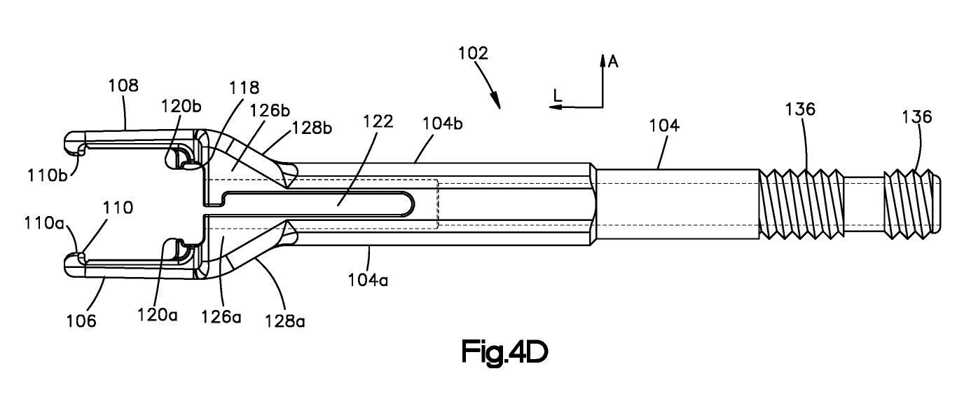

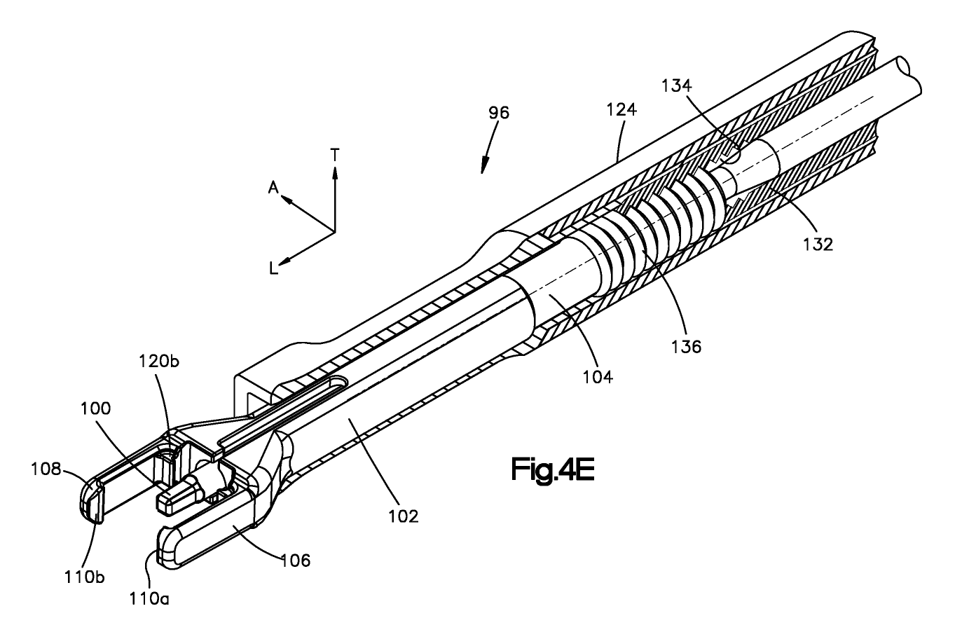

The intervertebral implant 20 defines a distal or leading end 28 and a proximal or trailing end 30 opposite the leading end 28 along a longitudinal direction L. As used herein, the term “distal” and derivatives thereof refer to a direction from the trailing end 30 toward the leading end 28. As used herein, the term “proximal” and derivatives thereof refer to a direction from the leading end 28 toward the trailing end 30. The distal and proximal directions can be oriented along the longitudinal direction L. The leading end 28 can also be referred to as an insertion end with respect to the direction of insertion of the implant 20 into the intervertebral space 22. Thus, the longitudinal direction L can define an insertion direction into the intervertebral space 22. The leading end 28 is spaced from the trailing end 30 in the insertion direction. In this regard, the insertion direction can be defined by the distal direction. In one example, the leading end 28 can be tapered and configured for insertion into the intervertebral space 22 between the first and second vertebral bodies 24 and 26. As will be described in more detail below, the trailing end 30 is configured to couple to an insertion instrument 96 shown in FIG. 4, which is configured to rigidly support and deliver the implant 20 into the intervertebral space 22, and iterate the implant 20 from a collapsed configuration shown in FIG. 2A to an expanded configuration shown in FIG. 2B. The implant 20 has a first height when in the collapsed configuration, and defines a second height when in the expanded configuration that is greater than the first height.

The intervertebral implant 20 includes a first or superior endplate 32 that defines a first or superior vertebral engagement surface 34 that is configured to abut the superior vertebral body 24, and a second or inferior endplate 36 that defines a second or inferior vertebral engagement surface 38 that is configured to abut the inferior vertebral body 26. In particular, the first and second endplates 32 and 36 of the implant 20 are configured to abut respective first and second vertebral endplates 25 and 27, respectively, of the superior and inferior vertebral bodies 24 and 26. The first and second vertebral endplates 25 and 27 can also be referred to as superior and inferior vertebral endplates 25 and 27, respectively. As used herein, the term “superior” and “up” and derivatives thereof refer to a direction from the second vertebral engagement surface 38 toward the first vertebral engagement surface 34. As used herein, the term “inferior” and “down” and derivatives thereof refer to a direction from the first vertebral engagement surface 34 toward the second vertebral engagement surface 38. The superior and inferior directions can be oriented along a transverse direction T. The first and second endplates 32 and 36, and thus the first and second vertebral engagement surfaces 34 and 38 are spaced from each other along the transverse direction T. The transverse direction T is oriented substantially perpendicular to the longitudinal direction L. In one example, the first and second endplates 32 and 36 can be configured to grip the first and second vertebral bodies, respectively. In one example, the first and second endplates 32 and 36 can have teeth 40 that project out from the vertebral engagement surfaces 34 and 38. The teeth 40 are configured to grip the superior and inferior vertebral bodies 24 and 26, respectively. In particular, the teeth 40 are configured to grip the superior and inferior vertebral endplates 25 and 27, respectively.

The intervertebral implant 20 is expandable from a collapsed position shown in FIG. 2A to an expanded position shown in FIG. 2B. Thus, the intervertebral implant 20 is configured to be inserted into the intervertebral disc space 22 in the collapsed configuration. The implant 20 is configured to be expanded from the collapsed configuration to the expanded configuration after the implant 20 has been implanted into the intervertebral space 22. Thus, a method can include the step of inserting the implant 20 into the intervertebral space 22 in a collapsed position, and subsequently iterating the implant 20 to the expanded position such that the first and second vertebral engagement surfaces 34 and 38 bear against the first and second vertebral endplates 25 and 27, respectively.

When the intervertebral implant 20 is in the collapsed configuration, the first and second vertebral engagement surfaces 34 and 38 are spaced apart a first distance along the transverse direction T. The first and second endplates 32 and 36 move apart from each other along the transverse direction T as the implant 20 moves from the collapsed configuration to the expanded configuration. In one example, respective entireties of the first and second endplates 32 and 36 are configured to move away from each other as the implant 20 expends from the collapsed position to the expanded position. When the intervertebral implant 20 is in the expanded configuration, the first and second vertebral engagement surfaces 34 and 38 are spaced apart a second distance along the transverse direction T that is greater than the first distance. Thus, the implant 20 is configured to impart appropriate height restoration to the intervertebral space 22. It should be appreciated that the implant 20 is configured to remain in the expanded configuration in the presence of compressive anatomical forces after implantation, and that the implant 20 is prevented from moving toward the collapsed position in response to the compressive anatomical forces. The intervertebral space 22 that receives the implant 20 can be disposed anywhere along the spine as desired, including at the lumbar, thoracic, and cervical regions of the spine.

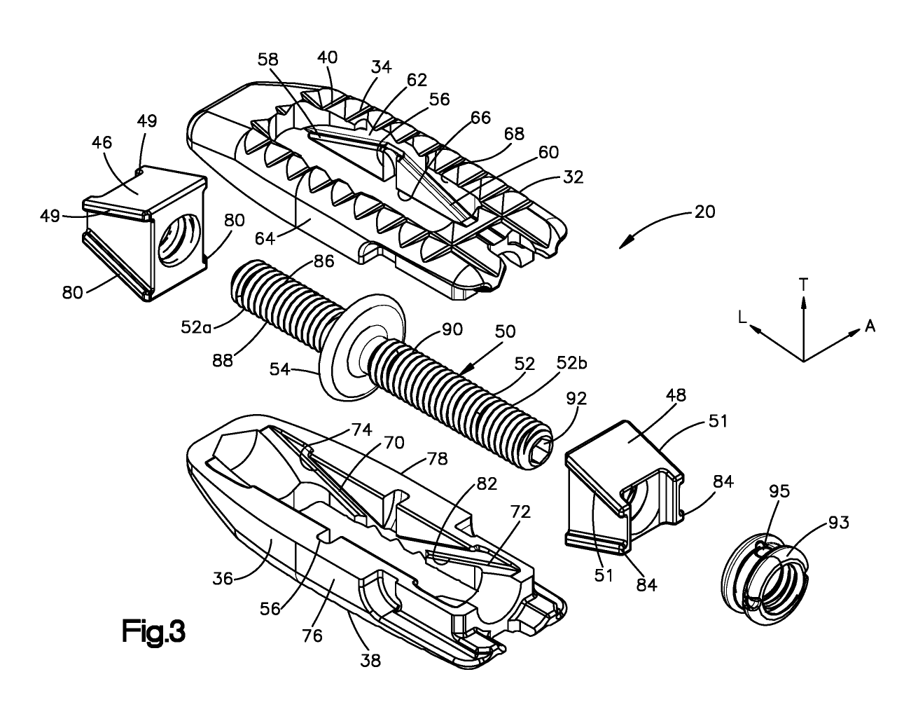

Referring now also to FIG. 3, the intervertebral implant 20 further includes at least one expansion member 42 that is configured to move between first and second positions that iterate the implant 20 between the collapsed configuration and the expanded configuration. The at least one expansion member 42 can include a first wedge member 46 and a second wedge member 48. The first and second wedge members 46 and 48 can be configured to couple the first and second endplates 32 and 36 to each other. The first and second wedge members 46 and 48 are translatable in a first direction along the longitudinal direction L so as to cause the first and second endplates 32 and 36 to move away from each other, thereby expanding the implant 20. The first and second wedge members 46 and 48 are translatable in a second direction along the longitudinal direction L opposite the first direction so as to cause the first and second endplates 32 and 36 to move toward from each other, thereby collapsing the implant 20.

The implant 20 can further include an actuator 50 coupled to the first and second wedge members 46 and 48. The actuator 50 includes a threaded actuator shaft 52 and an actuation flange 54 that protrudes from the actuator shaft 52. The actuation flange 54 fits into respective complementary slots 56 of the first and second endplates 32 and 36 so as to prevent the actuator 50 from translating relative to the endplates 32 and 36 along the longitudinal direction L.

The first endplate 32 defines first and second ramp surfaces 58 and 60 that are opposite the first vertebral engagement surface 34 along the transverse direction T. The first ramp surface 58 is angled in the superior direction as it extends in the proximal direction toward the second ramp surface 60. The second ramp surface 60 is angled in the superior direction as it extends in the distal direction toward the first ramp surface 58. The first wedge member 46 is configured to ride along the first ramp surface 58. Similarly, the second wedge member 48 is configured to ride along the second ramp surface 60.

The first ramp surface 58 can partially define a first ramped slot 62 in first and second side walls 64 and 66 of the first endplate 32 that are opposite each other along a lateral direction A that is perpendicular to each of the longitudinal direction L and the transverse direction T. The first wedge member 46 can define first upper rails 49 that are configured to ride in the first ramped slots 62. Thus, the first upper rails 49 are configured to ride along the first ramp surface 58. Similarly, the second ramp surface 60 can partially define a second ramped slot 68 in the first and second side walls 64 and 66. The second wedge member 48 can define second upper rails 51 that are configured to ride in the second ramped slots 68. Thus, the second upper rails 51 are configured to ride along the second ramp surface 60.

Similarly, the second endplate 36 defines first and second ramp surfaces 70 and 72 that are opposite the second vertebral engagement surface 38 along the transverse direction T. The first ramp surface 70 is angled in the inferior direction as it extends in the proximal direction toward the second ramp surface 72. The second ramp surface 72 is angled in the inferior direction as it extends in the distal direction toward the first ramp surface 70. The first wedge member 46 is configured to ride along the first ramp surface 70. Similarly, the second wedge member 48 is configured to ride along the second ramp surface 72.

The first ramp surface 70 can partially define a first ramped slot 74 in first and second side walls 76 and 78 of the second endplate 36 that are opposite each other along the lateral direction A. The first wedge member 46 can define first lower rails 80 that are configured to ride in the first ramped slots 74. Thus, the first lower rails 80 are configured to ride along the first ramp surface 70. Similarly, the second ramp surface 72 can partially define a second ramped slot 82 in the first and second side walls 76 and 78. The first side walls 64 and 76 can cooperate to define a first side 77 of the implant 20, and the second side walls 66 and 78 can cooperate to define a second side 79 of the implant 20. The second wedge member 48 can define second lower rails 84 that are configured to ride in the second ramped slots 82. Thus, the second lower rails 84 are configured to ride along the second ramp surface 72.

As the first and second wedge members 46 and 48 move in a first expansion direction, the first and second wedge members 46 and 48 push the first and second endplates 32 and 36 away from each other along the transverse direction T, thereby causing the implant 20 to expand along the transverse direction T. As the first and second wedge members 46 and 48 move in a second collapsing direction opposite the first expansion direction, the first and second wedge members 46 and 48 can draw the first and second endplates 32 and 36 toward each other along the transverse direction T, thereby collapsing the implant to collapse along the transverse direction T. The first expansion direction of the first and second wedge members 46 and 48 can be defined by movement of the first and second wedge members 46 and 48 toward each other. The second collapsing direction of the first and second wedge members 46 and 48 can be defined by movement of the first and second wedge members 46 and 48 away from each other. It should be appreciated, of course, that the implant can alternatively be constructed such that the first expansion direction of the first and second wedge members 46 and 48 can be defined by movement of the first and second wedge members 46 and 48 away each other, and the second collapsing direction of the first and second wedge members 46 and 48 can be defined by movement of the first and second wedge members 46 and 48 toward from each other.

With continuing reference to FIGS. 2A-3, the actuator 50 is configured to cause the first and second wedge members 46 and 48 to move in the first expansion direction. Further, the actuator 50 can be configured to cause the first and second wedge members 46 and 48 to move in the second collapsing direction. In particular, the actuator shaft 52 can be threaded so as to threadedly mate with the first and second wedge members 46 and 48, respectively. In one example, the actuator shaft 52 can define exterior threads 86. The actuation flange 54 can divide the actuator shaft 52 into a first or distal shaft section 52a and a second or proximal shaft section 52b.

The threads 86 can include a first threaded portion 88 that extends along the distal shaft section 52a, and a second threaded portion 90 that extends along the proximal shaft section 52b. The first wedge member 46 can include internal threads that are threadedly mated to the distal shaft section 52a. The second wedge member 48 can include internal threads that are threadedly mated to the proximal shaft section 52b. The first and second threaded portions 88 and 90 have respective thread patterns, respectively that are oriented in opposite directions. Accordingly, rotation of the actuator 50 in a first direction of rotation drives the wedge members 46 and 48 to threadedly travel away from each other along the actuator shaft 52. The actuator shaft 52 can be oriented along the longitudinal direction L. Thus, rotation of the actuator 50 in the first direction can cause the wedge members 46 and 48 to move in the expansion direction. Rotation of the actuator 50 in a second direction of rotation opposite the first direction of rotation drives the wedge members 46 and 48 to threadedly travel toward each other along the actuator shaft 52. Thus, rotation of the actuator 50 in the second direction can cause the wedge members 46 and 48 to move in the collapsing direction. The first and second directions of rotation can be about the central axis of the actuator shaft 52, which can be oriented along the longitudinal direction L.

The actuator 50, and thus the implant 20, can further include a driven member 92 that is rotationally fixed to the actuator shaft 52, such that a rotational force applied to the driven member 92 drives the actuator shaft 52, and thus the actuator 50, to rotate. The driven member 92 can be monolithic with the actuator shaft 52, and in one example can be defined by the actuator shaft 52. For instance, the driven member 92 can be configured as a socket that extends distally into the proximal end of the actuator shaft 52. Alternatively, the driven member 92 can be attached to the actuator shaft 52. The driven member 92 can be configured to couple to the insertion instrument 96 so as to receive a drive force that causes the actuator shaft 52, and thus the actuator 50, to rotate. In one embodiment, the driven member 92 can define a socket that is configured to receive a drive member of the insertion instrument 96. Alternatively, the driven member 92 can be configured to be received by the drive member.

The actuator 50, and thus the implant 20, can further include an implant coupler 93 that is supported by the driven member 92. In particular, the implant coupler 93 can be supported by the actuator shaft 52. The implant coupler 93 can be monolithic with the actuator shaft 52, or can be secured to the actuator shaft 52. For instance, the implant coupler 93 can be threadedly attached to the actuator shaft 52. In one example, the implant coupler 93 can be aligned with the driven member 92 along a plane that includes the lateral direction A and the transverse direction T. The implant coupler 93 can be configured to attach to a complementary attachment member of the insertion instrument 96. For instance, the implant coupler 93 can define an external groove 95 that is configured to receive the attachment member of the insertion instrument 96. The implant coupler 93 can be configured as a ring, or can be configured as any suitable alternatively constructed attachment member as desired. Aspects of the implant 20 are further described in U.S. patent application Ser. No. 14/640,264 filed Mar. 6, 2015, the disclosure of which is hereby incorporated by reference as if set forth in its entirety herein.

Referring to FIG. 4A-4B, an intervertebral implant system 94 can include the intervertebral implant 20 and an insertion instrument 96. The insertion instrument 96 can be configured to implant the expandable intervertebral implant 20 in the intervertebral space. For instance, the insertion instrument 96 can be configured to removably attach and further secure to the implant 20 so as to define a rigid construct with the implant 20. The insertion instrument 96 can further be configured to apply an actuation force to the actuator 50 that drives the actuator to rotate. For instance, the insertion instrument 96 can drive the actuator 50 to selectively rotate in the first direction of rotation and in the second direction of rotation.

Thus, a method can include the step of attaching the insertion instrument 96 to the intervertebral implant 20 to form a rigid construct. The implant 20 can initially be in the collapsed configuration when the insertion instrument 96 is coupled to the implant 20. Alternatively, the insertion instrument 96 can move the implant 20 to the collapsed position. The method can further include the step of actuating the drive member to rotate the actuator 50 of the implant 20 in the first direction of rotation, thereby causing the implant 20 to expand in the manner described above to a desired height. Once the implant 20 has achieved the desired height, the method can include the step of removing the insertion instrument 96 from the implant 20.

Referring now also to FIGS. 2A-3 and 4C-4E, the insertion instrument 96 can include a driver 97 that has a drive shaft 98 and a drive member 100. The drive shaft 98 is elongate along the longitudinal direction L. The drive shaft 98 can include a knob 99 at its proximal end that is configured to be gripped and rotated, to thereby rotate the drive shaft 98 about a longitudinal axis of rotation. The drive member 100 can be disposed at a distal end of the drive shaft 98. The drive member 100 can be monolithic with the drive shaft 98 or attached to the drive shaft 98. The drive member 100 is configured to couple to the driven member 92 (see FIG. 3). For instance, the drive member 100 and the socket defined by the driven member 92 can have a non-circular cross section. Accordingly, when the drive member 100 is inserted into the socket, rotation of the drive member 100 causes the actuator shaft 52 of the implant 20 to correspondingly rotate. Thus, it should be appreciated that rotation of the drive member 100 in the first direction of rotation causes the actuator 50 of the implant 20 to rotated in the first direction of rotation. Thus, the drive member 100 of the insertion instrument 96 can be configured to couple to the complementary driven member 92 of the implant 20, and iterate the intervertebral implant 20 from the collapsed configuration to the expanded configuration. Similarly, rotation of the drive member 100 in the second direction of rotation causes the actuator 50 of the implant 20 to rotated in the second direction of rotation. Thus, the drive member 100 can further iterate the intervertebral implant 20 from the expanded configuration to the collapsed configuration. As will be appreciated from the description below, the drive member 100 can be translated along the longitudinal direction between an extended position whereby the drive member 100 is positioned to be coupled to the driven member 92 when the insertion instrument 96 is attached to the implant 20, and a retracted position whereby the drive member 100 is removed from the driven member 92 when the insertion instrument 96 is attached to the implant 20.

The insertion instrument 96 can further include a securement member 102 that is configured to attach and secure to the implant 20. In particular, the securement member 102 is configured to iterate between an engaged configuration and a disengaged configuration. The securement member 102 is configured to attach to the implant 20 when in the disengaged configuration, and is secured to the implant 20 when in the engaged configuration. The securement member 102 is further configured to be removed from the implant 20 when in the disengaged configuration. The securement member 102 is configured to be prevented from removal from the implant 20 when the securement member 102 is in the engaged configuration, and thus when the securement member 102 is secured to the implant 20.

The securement member 102 can include a securement shaft 104 and a securement end 105 that extends distally from the securement shaft 104. The securement end 105 can include first and second securement plates 106 and 108 that extend from the securement shaft 104 in the distal direction. The first and second securement plates 106 and 108 can be spaced from each other along a direction perpendicular to the longitudinal direction L. For instance, the first and second securement plates 106 and 108 can be spaced from each other along the lateral direction A. The first and second securement plates 106 and 108 can be oriented parallel to each other. The first and second securement plates 106 and 108 can be positioned such that the drive member 100 extends between the first and second securement plates 106 and 108 along the lateral direction A. Further, the drive member 100 can be aligned with the first and second securement plates 106 and 108 along the lateral direction A.

The securement member 102 can further include at least one projection that can define at least one guide rail 110 that projects from a corresponding one of the first and second securement plates 106 and 108 toward the other of the first and second securement plates 106 and 108. The at least one guide rail is configured to slide along a respective at least one pair of side walls of the implant. The at least one pair can include a first pair 63 (see FIG. 2A) defined by the side walls 64 and 76 of the implant 20, and a second pair 65 (see FIG. 2A) defined by the side walls 66 and 78 of the implant 20. The implant 20 can define a first side 77 and a second side 79 that is opposite the first side 77 with respect to the lateral direction A. The first side 77 can be defined by the side walls 64 and 76 of the first pair 63. The second side 79 can be defined by the side walls 66 and 78 of the second pair 65. The first and second sides 77 and 79 are opposite each other along the lateral direction A. The side walls of each pair can be aligned with each other along the transverse direction T. Further, the side walls of each pair can abut each other when the implant is in the collapsed configuration.

The implant 20 can include at least one guide channel 112 that is defined by an outer surface of each of the pair of side walls of the implant 20. The at least one guide channel 112 is configured to receive the at least one first guide rail 110, such that the at least one guide rail 110 resides in the at least one guide channel 112 when the insertion instrument 96 is secured to the implant 20. The at least one guide rail 110 can also reside in the at least one guide channel 112 when the insertion instrument 96 is attached, but not secured, to the implant 20. The at least one guide rail 110 can have a height along the transverse direction T that is sufficient to 1) reside in the at least one guide channel 112 when the implant 20 is in the collapsed configuration, 2) ride along the implant 20 in the at least one guide channel 112 as the implant 20 expands to the expanded configuration, and 3) remain in the corresponding at least one guide channel 112 when the implant 20 is in the expanded configuration.

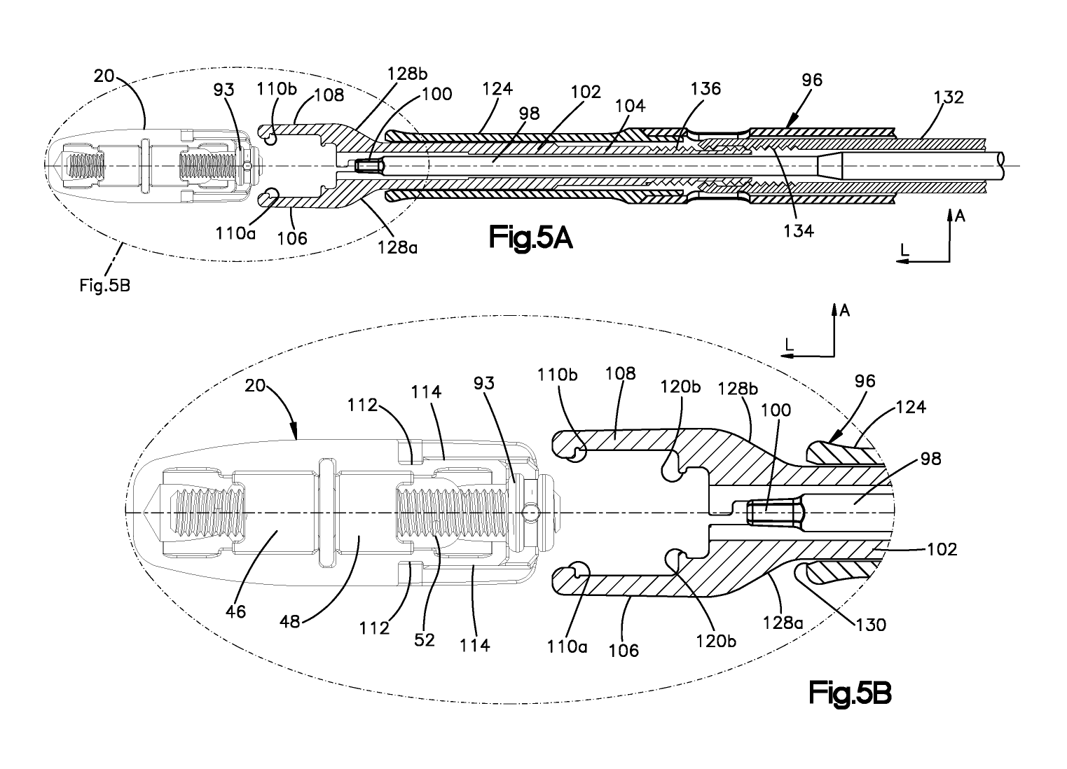

In one example, the securement member 102 can include a first guide rail 110a that projects from the first securement plate 106 toward the second securement plate 108, and a second guide rail 110b that projects from the second securement plate 108 toward the first securement plate 106. Thus, the first and second guide rails 110a and 110b can be spaced from each other along the lateral direction A, and can be inwardly facing. Further, the first and second guide rails 110a and 110b can be aligned with each other along the lateral direction A. The implant 20 can include a guide channel 112 that is defined by the outer surface of each of the first and second pairs 63 and 65 of side walls (see first and second guide channels 112 in FIG. 5B). Thus, the side walls 64 and 76 can each define a portion of a first guide channel 112. The side walls 66 and 78 can further define a portion of a second guide channel. The guide channel 112 of the first pair 63 of side walls is sized to receive the first guide rail 110a, and the guide channel 112 of the second pair 65 of side walls is sized to receive the second guide rail 110b.

The outer surface of the side walls of each of the first and second pairs 63 and 65 of side walls can further cooperate to define respective lead-in recesses 114 to the guide channel 112 (see first and second lead-in recesses 114 in FIG. 5B). The respective lead-in recess 114 is spaced in the proximal direction from the guide channel 112. For instance, each of the side walls of the implant 20 defines a corresponding portion of the respective lead-in recess. The respective endplates 32 and 36 can terminate the lead-in recesses 114 and the guide channels 112 along the transverse direction T. The guide channels 112 have a depth in the lateral direction A that is greater than the depth of the lead-in recesses 114 in the lateral direction A. As will be described in more detail below, the first and second guide rails 110a and 110b are configured to ride distally along the outer surface of the implant 20 in the respective lead-in recesses 114 and into the guide channels 112 when the insertion instrument 96 is in the disengaged configuration.

Because the securement plates 106 and 108 are resiliently supported by the securement shaft 104, and in particular by the first and second securement plates 106 and 108 respectively, and because the guide channels 112 are deeper than the lead-in recesses 114, the first and second guide rails 110a and 110b can resiliently move apart along the lateral direction as they cam over the implant 20, and can snap into the guide channels 112.

The distal end of the guide channels 112 can be defined by respective shoulders 116 that are defined by the respective side walls. The shoulders can protrude laterally outward with respect to the outer surface of the side walls at the lead-in recesses 114. Thus, the implant 20 defines a width along the lateral direction A at the guide channels 112 that is less than the width at the lead-in recesses 114. The width of the implant 20 at the lead-in recesses 114 is less than the width at the shoulders 116. The shoulders 116 provide stop surfaces configured to abut the guide rails 110a and 110b so as to prevent the guide rails 110a and 110b from traveling distally past the guide channels 112.

The first and second securement plates 106 and 108 define a height along the transverse direction T that is less than the height of the lead-in recesses 114 along the transverse direction T, both when the implant 20 is in the collapsed configuration and when the implant 20 is in the expanded configuration. Accordingly, the first and second securement plates 106 and 108 can reside in the lead-in recesses 114 when the first and second guide rails 110a and 110b are disposed in the respective guide channels 112. Further, in one example, the securement plates 106 and 108 have a width that is no greater than the depth of the lead-in recesses 114 with respect to the shoulders 116. Thus, the securement plates 106 and 108 can nest in the respective lead-in recesses 114. It is also appreciated in one example that the securement plates 106 and 108 are no wider along the lateral direction A, and no taller in the transverse direction T, than the intervertebral implant 20 when the implant 20 is in the collapsed configuration.

Further, the height of the first and second securement plates 106 and 108 can be greater than the distance between the respective pairs of side walls when the implant 20 is in the expanded configuration. Thus, the first and second guide rails 110a and 110b can remain inserted in the respective guide channels 112 when the implant 20 is in the expanded position. In one example, the first and second guide rails 110a and 110b can extend along respective entireties of the heights of the first and second securement plates 106 and 108, respectively. Alternatively, the first and second guide rails 110a and 110b can extend along respective portions less than the entireties of the heights of the first and second securement plates 106 and 108, respectively. In one example, the first and second guide rails 110a and 110b can have a height along the transverse direction T of between approximately 3 mm to approximately 7 mm, depending on the height of the intervertebral implant 20. In one narrow example, the height of the guide rails can be between approximately 3.7 mm and approximately 4 mm. As used herein, the terms “approximate” and “substantial” and derivatives thereof are used to account for variations in size and/or shape, such as may occur due to manufacturing tolerances and other factors.

The insertion instrument 96 can further include at least one instrument coupler 118 that is configured to attach to the implant coupler 93. For instance, the securement member 102 can include the at least one instrument coupler 118 that is configured to attach to the implant coupler 93 when the securement member 102 is in the disengaged configuration, and secure to the implant coupler 93 when the securement member 102 is in the engaged configuration. The at least one instrument coupler 118 can project from a corresponding one of the first and second securement plates 106 and 108 toward the other of the first and second securement plates 106 and 108. The at least one instrument coupler 118 is configured to be inserted into the external groove 95 of the implant coupler 93. For instance, the at least one attachment member can be configured to seat against the implant coupler 93 in the external groove 95 when the securement member 102 is in the engaged configuration.

The at least one instrument coupler 118 can be configured as a first collar 120a that projects from the first securement plate 106 toward the second securement plate 108, and a second collar 120b that projects from the second securement plate 108 toward the first securement plate 106. Each of the first and second collars 120a and 120b are configured to be inserted into the external groove 95 of the implant coupler 93 when the securement member 102 is in the disengaged configuration, and secured to the implant coupler 93 in the external groove 95 when the securement member 102 is in the engaged configuration. In particular, the first and second collars 120a and 120b can cam over the implant coupler 93 and snap into the groove 95 as the insertion instrument 96 is attached to the implant 20. In particular, when the insertion instrument 96 is in the disengaged configuration, the first and second collars 120a and 120b can be spaced from each other along the lateral direction A a distance that is less than the width of a portion of the implant coupler 93 that is disposed proximally from the external groove 95. Because the first and second collars 120a and 120b are resiliently supported by the securement shaft 104, and in particular by the first and second securement plates 106 and 108 respectively, the first and second collars 120a and 120b can resiliently move apart along the lateral direction A as they cam over the portion of the implant coupler 93, and can snap toward each other once they have cleared the portion of the implant coupler and travel into the external groove 95.

The first and second collars 120a and 120b can be aligned with each other along the lateral direction A. Further, at least a portion of each of the first and second collars 120a and 120b is aligned with a portion of the drive member 100 along the lateral direction A when the drive member 100 is in the engaged position. The collars 120a-b can be positioned such that the drive member 100 is disposed between the guide rails 110a-b and the collars 120a-b with respect to the longitudinal direction L when the drive member 100 is in the extended position.

As described above, the first and second securement plates 106 and 108 can be resiliently supported by the securement shaft 104. For instance, in one example, the securement shaft 104 can be forked so as to define first and second securement shaft portions 104a and 104b spaced from each other along the lateral direction A, and separated from each other by a slot 122. Thus, the first and second securement shaft portions 104a and 104b are resiliently movable with respect to each other along the lateral direction A. The first securement plate 106 can extend distally from the first securement shaft portion 104a, and the second securement plate 108 can extend distally from the second securement shaft portion 104b. Accordingly, the first and second securement plates 106 and 108 are resiliently movable with respect to each other along the lateral direction A. Thus, it should be appreciated that the first and second guide rails 110a and 110b are resiliently movable with respect to each other along the lateral direction A. Further, the first and second collars 120a and 120b are resiliently movable with respect to each other along the lateral direction A.

When the securement member 102 is in an initial position the first and second securement plates 106 and 108 are spaced from each other a first distance along the lateral direction A. In the initial position, the securement member 102 is in the disengaged configuration whereby the securement member is configured to be attached to, or removed from, the implant 20. The securement member 102 is configured to receive a biasing force that urges the securement plates 106 and 108 toward each other along the lateral direction A, such that the securement plates 106 and 108 are spaced from each other a second distance along the lateral direction A that is less than the first distance. The securement member 102 thus iterates to the engaged position in response to the biasing force, whereby the securement member 102, and thus the insertion instrument 96, is configured to be secured to the implant 20. Accordingly, the biasing force can urge the first and second guide rails 110a and 110b into the respective guide channels 112. Similarly, the biasing force can urge the first and second collars 120a and 120b into the groove 95 of the driven member 92. It is recognized that increased biasing forces increases the securement of the securement member 102 to the implant 20, and thus of the insertion instrument 96 to the implant 20.

With continuing reference to FIGS. 2A-3 and 4C-4E, the insertion instrument 96 can further include a biasing member 124. As will be appreciated from the description below, the securement member 102 is movable with respect to the biasing member 124 between an engaged position and a disengaged position. When the securement member 102 is in the engaged position, the biasing member 124 delivers the biasing force to the securement member 102. The biasing force can cause the securement member 102 to iterate to the engaged configuration. When the securement member 102 is in the disengaged position, the biasing member 124 removes the biasing force from the securement member 102, thereby causing the securement member 102 to be in the relaxed disengaged configuration. The movement of the securement member 102 between the engaged position and the disengaged position can be along the longitudinal direction L.

The securement member 102 can include at least one bearing member that is in mechanical communication with the first and second securement plates 106 and 108. For instance, the at least one bearing member can extend from the first and second securement plates 106 and 108 such that the biasing force can be applied to the bearing member that, in turn, urges the first and second securement plates toward each other, thereby iterating the securement member 102 to the engaged configuration. The at least one bearing member can include first and second bearing members 126a and 126b that are spaced from each other along the lateral direction A. The biasing member 124 is configured to bear against the bearing members 126a and 126b as the securement member 102 travels toward the engaged position, such that the biasing member 124 applies the biasing force to the bearing members 126a and 126b.

The first and second bearing member 126a can extend between the securement shaft 104 and the first securement plate 106, and the second bearing member 126b can extend between the securement shaft 104 and the second securement plate 108. For instance, the first bearing member 126a can extend between the first securement shaft portion 104a and the first securement plate 106. The second bearing member 126b can extend between the second securement shaft portion 104b spaced and the second securement plate 108. The first bearing member 126a can define a first bearing surface 128a that flares away from the second bearing member 126b as it extends toward the first securement plate 106. Similarly, the second bearing member 126b can define a second bearing surface 128b that flares away from the first bearing member 126a as it extends toward the second securement plate 108. Thus, the first and second bearing surfaces 128a and 128b can flare away from each other each other as they extend toward the first and second securement plates 106 and 108, respectively.

As the securement member 102 travels from the disengaged position to the engaged position, the biasing member 124 bears against one or both of the first and second bearing surfaces 128a and 128b, thereby applying a biasing force that urges the bearing surfaces 128a and 128b toward each other along the lateral direction A. As a result, the first and second bearing members 126a and 126b are urged toward each other along the lateral direction A, which in turn urges the first and second securement plates 106 and 108 to move toward each other along the lateral direction A.

In particular, the biasing member 124 can include respective biasing surfaces 130 at its distal end. The biasing surfaces 130 are aligned with the bearing surfaces 128a and 128b along the longitudinal direction L. Thus, as the securement member 102 travels relative to the biasing member 124 toward the engaged position, the biasing surfaces 130 are brought into contact with the respective first and second bearing surfaces 128a and 128b, thereby causing the biasing force to be applied to the securement plates 106 and 108. Further movement of the securement member 102 with respect to the biasing member 124 toward the engaged position causes the biasing surfaces 130 to travel distally along the outwardly tapered bearing surfaces 128a and 128b. The distal travel of the biasing surfaces 130 along the first and second bearing surfaces 128a and 128b causes the biasing forces to increase. The biasing force can be sufficient to retain the first and second guide rails 110a and 110b in the respective first and second guide channels 112 of the implant 20 both when the implant 20 is in the collapsed configuration and when the implant 20 is in the expanded configuration. Further, the biasing force can be sufficient to retain the collars 120a and 120b in the external groove 95 of the implant coupler 93.

It is appreciated that movement of the securement member 102 in the proximal direction with respect to the biasing member 124 moves the securement member 102 toward the engaged position. Movement of the securement member 102 in the distal direction with respect to the biasing member 124 moves the securement member 102 toward the disengaged position, whereby the biasing surfaces 130 move proximally along the inwardly tapered bearing surfaces 128a and 128b. Proximal movement of the biasing surfaces 130 with respect to the bearing surfaces 128a and 128b causes the biasing forces to decrease until the biasing surfaces 130 are removed from the bearing surfaces 128a and 128b.

The insertion instrument 96 can further include an engagement member 132 that is configured to engage the securement member 102 so as to cause the securement member 102 to travel with respect to the biasing member 124. In particular, the engagement member 132 can include threads 134, and the securement member 102 can similarly include threads 136 that threadedly mate with the threads 134 of the engagement member 132. The threads 136 can be divided into proximal and distal threaded segments that are spaced from each other by a gap. The gap can have a length along the longitudinal direction L that is greater than the length of the threads 134 along the longitudinal direction. Thus, as will be described in more detail below, the threads 134 can become captured in the gap, such that relative rotation between then engagement member 132 and the securement member 102 will not cause relative translation until the threads 134 and 136 are engaged. The securement member 102 can extend into the engagement member. Thus, the threads 134 of the engagement member 132 can be internal threads, and the threads 136 of the securement member 102 can be external threads that are defined by the securement shaft 104. Accordingly, rotation of the engagement member 132 in a first direction of rotation with respect to the securement member 102 causes the securement member 102 to translate proximally with respect to the biasing member 124 toward the engaged position. Rotation of the engagement member 132 in a second direction of rotation opposite the first direction of rotation causes the securement member 102 to translate distally with respect to the biasing member 124 toward the disengaged position. The engagement member 132 and the biasing member 124 can be translatably fixed to each other with respect to relative translation along the longitudinal direction L. Accordingly, translation of the securement member 102 with respect to the engagement member 132 is also with respect to the biasing member 124. The engagement member 132 can include a knob 138 at its proximal end that can be grasped by a user to facilitate rotation of the engagement member 132. The insertion instrument 96 can further include a handle 131 that is fixedly attached to the biasing member 124 with respect to relative translation along the longitudinal direction. In one example, the handle 131 can be rigidly fixed to the biasing member 124. For instance, the handle 131 can be attached to the biasing member 124 or can be monolithic with the biasing member 124. Thus, as the user grasps and holds the handle 131, the biasing member 124 can remain stationary while the securement member 32 translates relative to the biasing member 124.

The securement member 102 can be prevented from rotating as the engagement member 132 is rotated. In particular, the securement shaft 104 can define an outer surface that is non-circular, and the biasing member 124 can define an inner surface that is non-circular and contacts the non-circular outer surface of the securement shaft 104. The non-circular surfaces can engage so as to prevent relative rotation between the securement shaft 104 and the biasing member 124. Thus, the securement member 102 is rotatably fixed to the biasing member 124. Accordingly, rotation of the engagement member 132 does not cause the securement member 102 to correspondingly rotate with respect to the biasing member 124. As a result, the first and second securement plates 106 and 108 can remain spaced from each other along the lateral direction A.

The insertion instrument 96 can be arranged such that the engagement member 132 extends into the biasing member 124, and the securement member 102 extends into both the biasing member 124 and the engagement member 132. For instance, the proximal end of the securement member 102 can extend into the distal end of the engagement member 132. The drive shaft 98 can extend through the engagement member 132 and the securement member 102, such that the drive member 100 can extend to a location between and aligned with the first and second securement plates 106 and 108 with respect to the lateral direction A. The drive shaft 98 can translate proximally and distally with respect to each of the engagement member 132 and the securement member 102.

Operation of the intervertebral implant system 94 will now be described with reference to FIGS. 5A-11. In particular, referring initially to FIGS. 5A-5B, the insertion instrument 96 can be aligned with the implant 20 along the longitudinal direction L while the securement member 102 is in the disengaged configuration. The implant 20 is in the collapsed configuration. When the insertion instrument 96 is aligned with the implant 20 along the longitudinal direction L, the first guide rail 110a can be substantially aligned with the first pair of side walls 64 and 76 along the longitudinal direction L, and the second guide rail 110b can be substantially aligned with the second pair 65 of side walls 66 and 78 along the longitudinal direction L. For instance, the first securement plate 106, and thus the first guide rail 110a, can be substantially aligned with the lead-in recess 114 at the first side 77 of the implant 20 along the longitudinal direction L. The second securement plate 108, and thus the second guide rail 110b, can be substantially aligned with the lead-in recess 114 at the second side 79 of the implant 20 along the longitudinal direction L. Further, the first and second collars 120a and 120b can be aligned with opposite sides of the implant coupler 93 of the implant 20 along the longitudinal direction L.

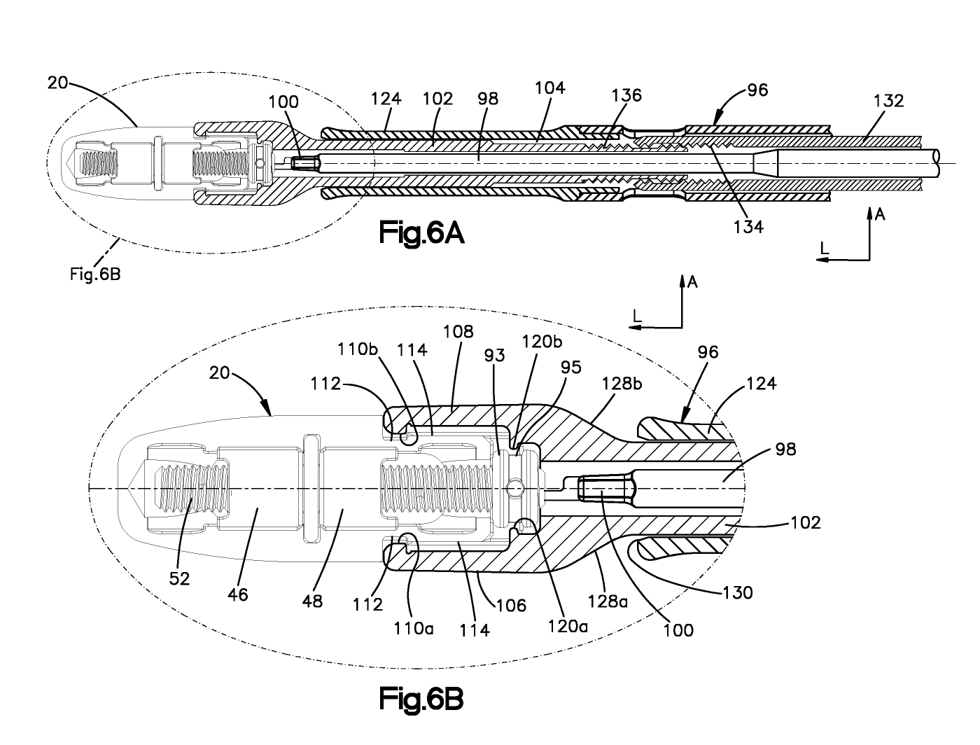

Referring now to FIGS. 6A-6B, the insertion instrument 96 can be advanced distally with respect to the implant 20 so as to removably attach the insertion instrument 96 to the implant 20. This advancement of the insertion instrument 96 relative to the implant 20 can be achieved by moving the insertion instrument 96 distally, or by moving the implant 20 proximally, or both. As the insertion instrument 96 is advanced distally relative to the implant 20, the first and second guide rails 110a and 110b ride along the first and second sides 77 and 79 of the implant 20, respectively, in the respective lead-in recesses 114. The distance between the first and second guide rails 110a and 110b along the lateral direction A when the securement member 102 is in the disengaged configuration can be less than the width of the implant 20 at the lead-in recesses 114. Thus, the first and second securement plates 106 and 108 can flex outward away from each other as the first and second guide rails 110a and 110b ride distally along the first and second sides 77 and 79 of the implant 20 in the lead-in recesses 114. The insertion instrument 96 is advanced distally 96 until the first and second guide rails 110a and 110b are inserted into the respective guide channels 112 of the first and second sides 77 and 79 of the implant 20. When the first and second guide rails 110a and 110b are inserted into the respective guide channels 112, the first and second securement plates 106 and 108 can nest in the respective lead-in recesses 114.

Similarly, the distance between the first and second collars 120a and 120b along the lateral direction A when the securement member 102 is in the disengaged configuration can be less than the width of the implant coupler 93. The implant coupler 93 can have a circular cross-section such that the width is a diameter, though the implant coupler 93 can have any suitable size and shape. Thus, as the first and second securement plates 106 and 108 flex outward away from each other, the first and second collars 120a and 120b ride distally along opposed sides of the implant coupler 93 until the first and second guide couplers 120a and 120b are inserted into the external groove 95. With the guide rails 110a and 110b received in the guide channels 112 and with the collars 120a and 120b received in the groove 95, the insertion instrument 96 can be said to be attached to the implant 20. It should be appreciated that when the insertion instrument 96 is attached to the implant 96, the spring constant defined by the resiliently deflected first and second securement plates 106 and 108 provides an attachment force that maintains the attachment of the insertion instrument to the implant 96. The insertion instrument 96 can be removed from the instrument by moving the insertion instrument 96 proximally with respect to the implant 20 so as to overcome the attachment force.

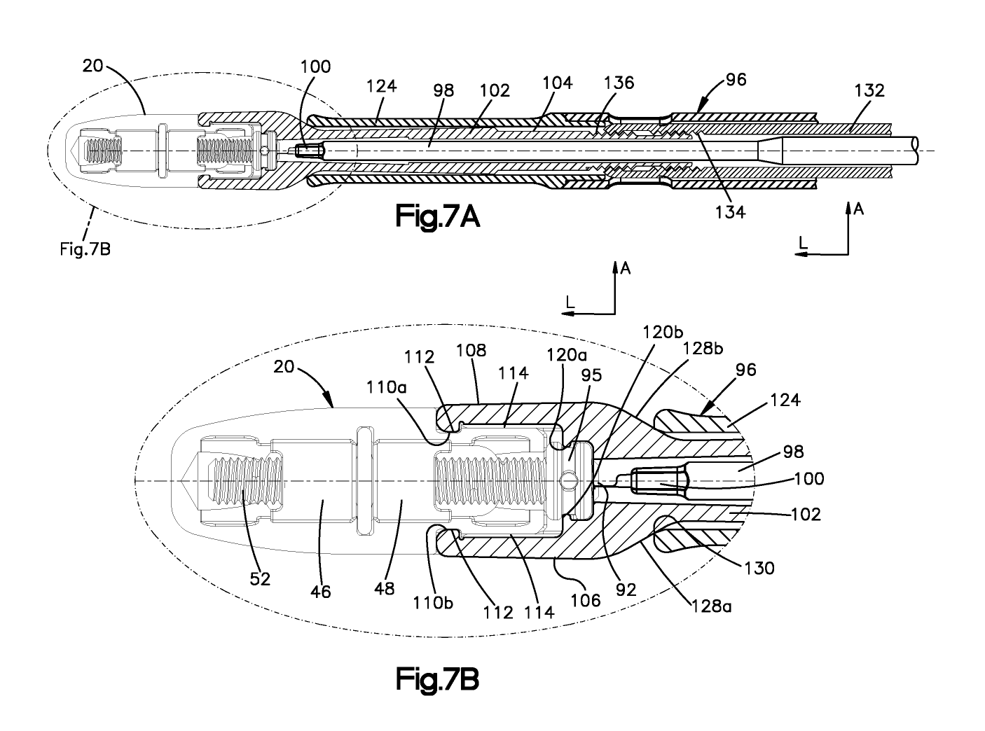

Referring now to FIGS. 7A-7B, the insertion instrument 96 can be secured to the implant 20 to define a rigid construct with the implant 20. In particular, the engagement member 132 can be rotated in the first direction of rotation with respect to the securement member 102, thereby causing the securement member 102 to translate with respect to the biasing member 124 toward the engaged position. The securement member 102 translates proximally until the biasing member 124 applies the biasing force to the securement member 102 in the manner described above. In particular, the biasing member 124 can apply the biasing force to the first and second bearing members 126a and 126b. The biasing force increases as the securement member 102 translates in the proximal direction while the biasing member 124 is in contact with the bearing members 126a and 126b. As the biasing force increases, the securement plates 106 and 108, including the alignment rails 110a-b, are urged against the implant 20 with increasing force, thereby increasing the rigidity of the construct defined by the insertion instrument 96 and the implant 20. The collars 120a-b can be seated in the groove without contacting the outer surface of the implant coupler 93. Thus, the collars 120a-b can be captured by the implant coupler 93 with respect to the longitudinal direction L so as to attach the collars 120a-b to the implant coupler 93. It should be appreciated that the collars 120a-b can remain attached to the implant coupler 93 both when the implant 20 is in the collapsed configuration and when the implant 20 is in the expanded configuration.

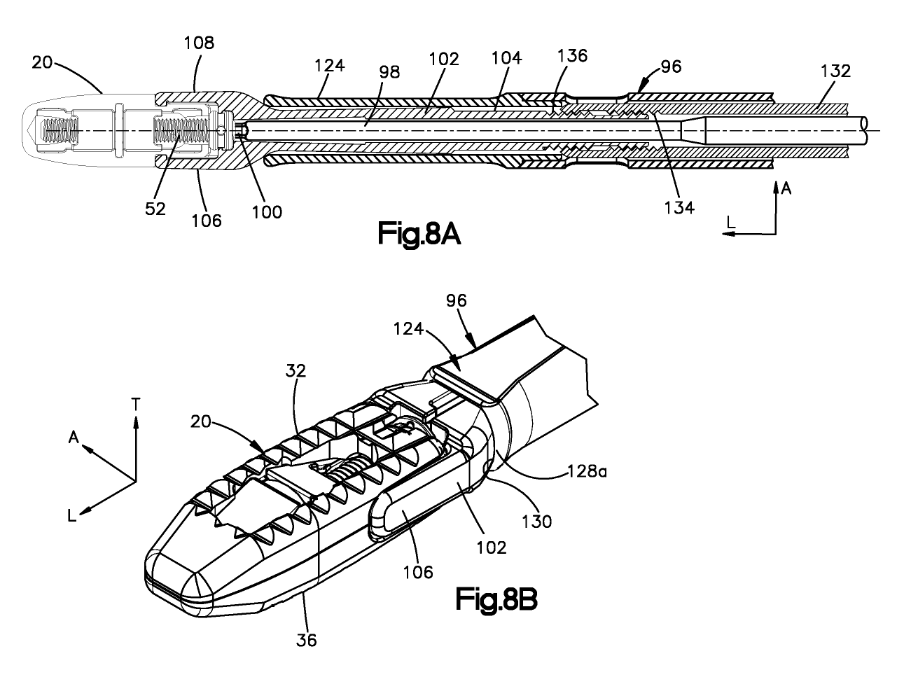

Referring now to FIGS. 8A-8B, the drive shaft 98 can be advanced distally until the drive member 100 is rotatably coupled to the driven member 92. For instance, the drive member 100 can be inserted into the driven member 92. Alternatively, the drive member 100 can be received by the driven member 92. It should be appreciated that the step of rotatably coupling the drive shaft 98 to the driven member 92 can be performed before, after, or during securement of the insertion instrument 96 to the implant 20. Further, the step of rotatably coupling the drive shaft 98 to the driven member 92 can be performed before or after the insertion instrument 96 is attached to the implant 20. When the drive member 100 is coupled to the driven member 92, it is recognized that the insertion instrument 96 is attached and secured to the implant 20 at three different attachment and securement locations. A first attachment and securement location is defined by the insertion of the guide rails 110a-b in to the guide slots 112, a second attachment and securement location is defined by the insertion of the collars 120a-b into the groove 95, and a third attachment and securement location is defined by the attachment of the drive member 100 to the driven member 92. When the insertion instrument 96 is secured to the implant 20, the insertion instrument 96 can deliver the implant 20 into the intervertebral space 22 (see FIG. 1).

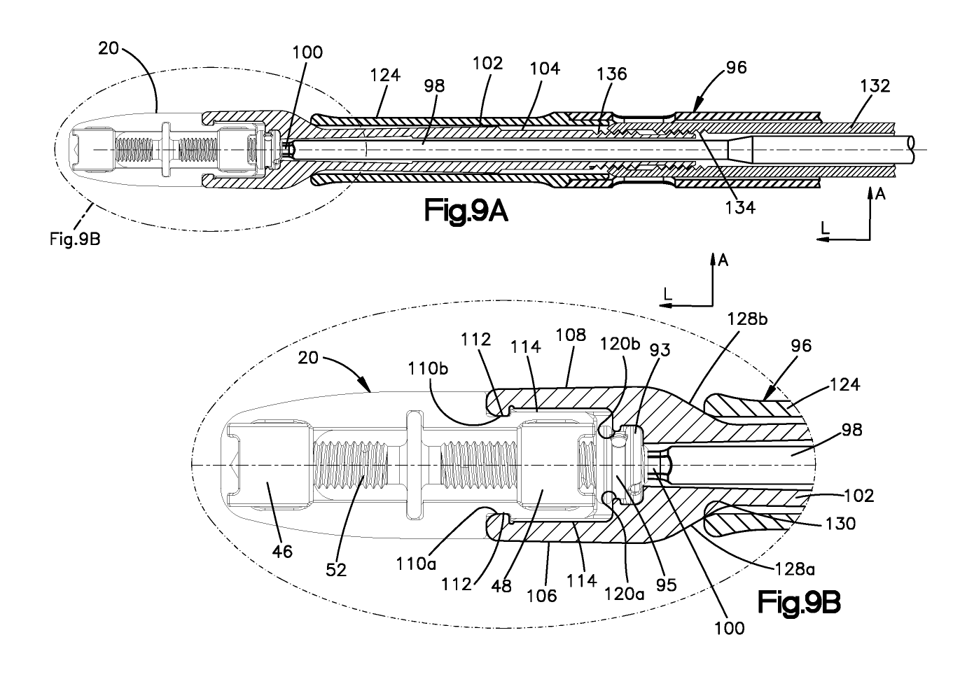

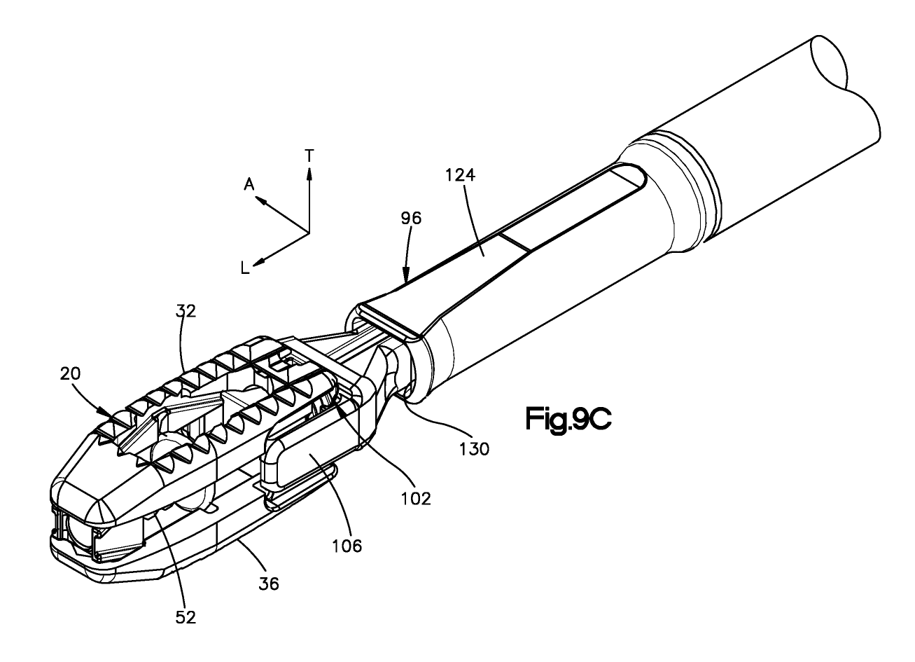

Referring now to FIGS. 9A-9C, when the insertion instrument 96 is secured to the implant 20 and the drive member 100 is coupled to the driven member 92, the drive member 100 can be rotated in the first direction of rotation so as to cause the implant 20 to expand from the collapsed configuration to the expanded configuration as described above. It should be appreciated that the first direction of rotation of the drive member 100 can be the same direction as the first direction of rotation of the engagement member 132. Alternatively, the first direction of rotation of the drive member 100 can be in an opposite direction with respect to the first direction of rotation of the engagement member 132. As the drive member 100 rotates in the first direction of rotation, the first and second wedge members 46 and 48 move in the expansion direction, so as to cause the first and second endplates 32 and 36 to translate away from each other in the manner described above.

When the implant 20 is in the expanded position, the first and second pairs 63 and 65 of side walls can separate from each other so as to define a gap therebetween. The first and second securement plates 106 and 108 can have a height sufficient to span the gap and remain the respective portions of the lead-in recess 114 defined by the respective side walls of each pair of side walls when the implant 20 is in the expanded position. Similarly, the guide rails 110a and 110b can have a height sufficient to span the gap and remain in respective portions of the guide slots 112 defined by the respective side walls of each pair of side walls when the implant 20 is in the expanded position. The guide rails 110a-110b can ride in the guide slots 112 along the transverse direction T as the implant 20 expands to the expanded position. Similarly, the securement plates 106 and 108 can ride in the lead-in recesses 114 along the transverse direction T as the implant 20 expands to the expanded position. In this regard, it is appreciated that increased biasing forces can cause the instrument 20 add increase resistance to the expansion of the implant 20.—please add a detail about 120a and 120b holding on slot 95 in 93 as an additional means of engagement regardless of how far opened or closed the cage is.

If it is desired to move the implant from the expanded configuration toward the collapsed configuration, the drive member 100 can be rotated in the second direction of rotation, thereby causing the wedge members 46 and 48 to move in the collapsing direction as described above.

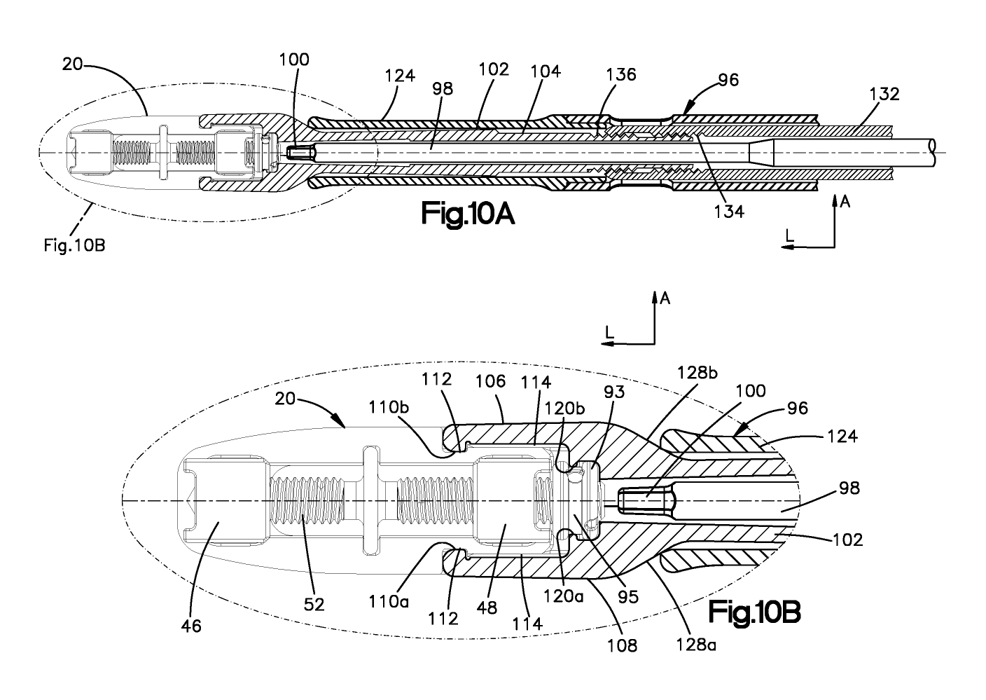

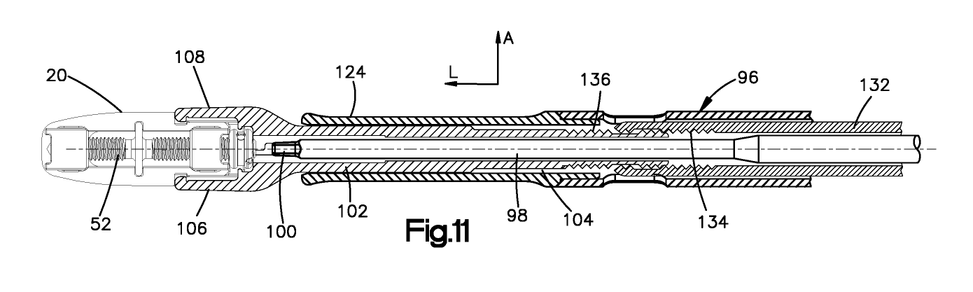

Referring now to FIGS. 10A-11, once the implant 20 has reached a desired height in the intervertebral space, the insertion instrument 96 can be removed from the implant 20. In particular, as illustrated in FIGS. 10A-10B, the securement member 102 can iterate from the engaged configuration to the disengaged configuration. In particular, the engagement member 132 can be rotated in the respective second direction of rotation with respect to the securement member 102, thereby causing the securement member 102 to travel with respect to the biasing member 124 toward the disengaged position. As described above, travel of the securement member 102 in the distal direction can be toward the disengaged position. As the securement member 102 travels with respect to the biasing member 124 to the disengaged position, the biasing member 124 removes the biasing force from the securement member 102. The engagement member 132 can be rotated until the threads 134 of the engagement member 132 are disengaged from the distal threaded segment of the threads 136 of the securement member 102, and captured in the gap that extends between the proximal and distal threaded segments of the threads 136. Accordingly, the engagement member 132 is preventing from rotating a sufficient amount that would inadvertently detach the securement member 102 from the engagement member 132. Rather, once the threads 134 are disposed in the gap, the engagement member 132 can be pulled distally with respect to the securement member so as to engage the threads 134 with the proximal segment of the threads 136. The engagement member 132 can then be rotated with respect to the securement member 102 so as to detach the securement member from the engagement member 132. Alternatively, the entire length of the threads 136 can be continuous and uninterrupted along the longitudinal direction L. Alternatively still, the threads 134 can be divided into proximal and distal segments that are configured to capture the threads 136 therebetween.

Referring to FIG. 11, the drive member 100 can be rotatably decoupled from the driven member 92. Thus, rotation of the drive member 100 does not cause the drive member 92 to rotate. In one example, the drive member 100 can be translated proximally so as to rotatably decouple from the driven member 92. It should be appreciated that the drive member can be rotatably decoupled from the driven member 92 before, after, or during movement of the securement member 102 with respect to the biasing member 124 to the disengaged position. Finally, the insertion instrument 96 can be moved proximally with respect to the implant 20 so as to entirely remove the insertion instrument 96 from the implant 20 as illustrated in FIGS. 5A-5B. In particular, the securement plates 106 and 108 are removed from the lead-in recesses 114.

It should be appreciated that the insertion instrument 96 has been described in accordance with one embodiment whereby the securement member 102 is configured to travel along the longitudinal direction L so as to iterate the securement member 102 between the engaged configuration and the disengaged configuration. Movement of the securement member 102 relative to the biasing member 124 causes the biasing member to apply and release the biasing force. It should be appreciated in alternative embodiments that the biasing member 124 can alternatively travel along the longitudinal direction L and the securement member 102 can remain stationary. In this alternative embodiment, relative travel exists between the securement member 102 and the biasing member 124. Thus, in this alternative embodiment, it can be said that the securement member 102 travels with respect to the biasing member 124, thereby causing the securement member 102 to iterate between the engaged configuration and the disengaged configuration in the manner described above.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. An insertion instrument configured to implant an expandable intervertebral implant in an intervertebral space, the insertion instrument comprising:
a drive shaft elongate along a longitudinal direction;
a drive member disposed at a distal end of the drive shaft and configured to 1) couple to a complementary driven member of the implant, and 2) iterate the intervertebral implant from a collapsed configuration to an expanded configuration; and
a securement member spaced from the drive member along a lateral direction that is perpendicular to the longitudinal direction, the securement member having at least one guide rail that has a height along a transverse direction sufficient to 1) reside in a corresponding at least one guide channel of both an inferior endplate and a superior endplate of the implant when the implant is in the collapsed configuration, 2) ride along the implant in the at least one guide channel as the implant expands to the expanded configuration, and 3) remain in the corresponding at least one guide channel of both the inferior endplate and the superior endplate when the implant is in the expanded configuration,
wherein the transverse direction is perpendicular to each of the longitudinal direction and the lateral direction.

2. The inserter of claim 1, further comprising a collar that is configured to be inserted in a corresponding groove of a coupler of the implant that is supported by the driven member while the drive member is engaged with the driven member.

3. The inserter of claim 2, wherein at least a portion of the collar is aligned with a portion of the drive member along the lateral direction.

4. The inserter of claim 3, wherein the drive member is disposed between the at least one guide rail and the collar with respect to the longitudinal direction.

5. The inserter of claim 1, wherein the securement member comprises first and second securement plates, and the at least one guide rail comprises a first guide rail that projects from the first securement plate toward the second securement plate, and a second guide rail the projects from the second securement plate toward the first securement plate.

6. The inserter of claim 5, wherein the drive member extends between the first and second securement plates along the lateral direction.

7. The inserter of claim 5, wherein the first and second securement plates have respective heights along the transverse direction, and the first and second guide rails extend along respective entireties of the heights of the first and second securement plates, respectively.

8. The inserter of claim 5, further comprising a biasing member configured to travel along the securement member between an engaged position whereby the biasing member applies a biasing force to the first and second securement plates that urge the first and second securement plates toward each other along the lateral direction, and a disengaged position wherein the biasing force is removed from the first and second securement plates.

9. The inserter of claim 8, wherein the biasing force is sufficient to retain the first and second guide rails in respective first and second guide channels of the implant both when the implant is in the collapsed configuration and when the implant is in the expanded configuration.

10. The inserter of claim 9, wherein the securement member comprises opposed first and second bearing members that are spaced from each other along the lateral direction and extend from the first and second securement plates, respectively, and the biasing member is configured to bear against the bearing members as it travels toward the engaged position, such that the biasing force is applied to the bearing members.

11. The inserter of claim 10, wherein the first and second bearing members define respective first and second bearing surfaces that flare away from each other each other as they extend toward the first and second securement plates, respectively, and the biasing member is configured to bear against the bearing surfaces as it travels toward the engaged position, such that the biasing force is applied to the bearing surfaces.

12. The inserter of claim 9, further comprising an engagement member that is threadedly mated with the biasing member, such that relative rotation between the engagement member and the biasing member in a first direction causes the biasing member to travel along the securement member toward the engaged position, and relative rotation between the engagement member and the biasing member in a second direction opposite the first direction causes the biasing ember to travel along the securement member toward the disengaged position.

13. The inserter of claim 12, wherein the drive shaft extends into both the engagement member and the securement member.

14. The inserter of claim 13, wherein the engagement member extends into the biasing member.

15. The inserter of claim 9, wherein the securement member comprises a securement shaft, such that the first and second securement plates extend from the securement shaft, wherein the first and second securement plates are resiliently forked so as to be naturally spaced apart a first distance when the biasing member is in the disengaged position, and the first and second securement plates are spaced apart a second distance less than the first distance when the biasing member is in the engaged position.

16. The inserter of claim 9, wherein the securement member further comprises at least one collar that extends from at least one of the first and second securement plates toward the other of the first and second securement plates, wherein the collar is configured to seat in a groove of the driven member.

17. The inserter of claim 16, wherein the collar includes a first collar that extends from the first securement plate toward the second securement plate, and a second collar that extends from the second securement plate toward the first securement plate.

18. The inserter of claim 17, wherein the biasing force is further configured to urge the first and second collars into the groove of the driven member.

19. An intervertebral implant system comprising:
the inserter of claim 1; and
the intervertebral implant of claim 1.

20. The intervertebral implant system as recited in claim 19,
wherein the securement member comprises first and second securement plates, and the at least one guide rail comprises a first guide rail that projects from the first securement plate toward the second securement plate, and a second guide rail the projects from the second securement plate toward the first securement plate, and
wherein the securement plates are no wider or taller than the intervertebral implant when the implant is in the collapsed configuration.
